(19) United States
(12) Patent Application Publication
(10) Pub. No.: US 2010/0249860 A1
(43) Pub. Date: Sep. 30, 2010

(54) EXTERNAL CARDIAC STIMULATION PATCH

(76) Inventors: Allan C. Shuros, St. Paul, MN (US); Eric A. Mokelke, White Bear Lake, MN (US); James A. Esler, Coon Rapids, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/720,246

(22) Filed: Mar. 9, 2010

Related U.S. Application Data
(60) Provisional application No. 61/162,809, filed on Mar. 24, 2009.

(51) Int. Cl. A61N 1/365; A61N 1/39

(52) U.S. Cl. 607/4; 607/10

(57) ABSTRACT

An external cardiac stimulation patch integrates a transcutaneous cardiac stimulation device and body-surface electrodes with a skin patch. The skin patch is to be attached onto a patient to provide for electrical contacts between the body-surface electrodes and a patient. The transcutaneous cardiac stimulation device delivers pacing pulses to the heart of the patient through pacing electrodes selected from the body-surface electrodes.

### CROSS-REFERENCE TO RELATED APPLICATION

This application claims the benefit of U.S. Provisional Application No. 61/162,809, filed on Mar. 24, 2009, under 35 U.S.C. §119(e), which is hereby incorporated by reference.

This application is related to co-pending, commonly assigned, U.S. patent application Ser. No. 61/079,008, entitled “METHOD AND APPARATUS FOR TRANSCUTANEOUS CARDIOPROTECTIVE PACING”, filed on Jul. 8, 2008, which is hereby incorporated by reference in its entirety.

### TECHNICAL FIELD

This document relates generally to cardiac stimulation systems and particularly to an external cardiac stimulation patch for transcutaneous delivery of pacing pulses to the heart.

### BACKGROUND

The heart is the center of a person's circulatory system. It includes an electro-mechanical system performing two major pumping functions. The left portions of the heart draw oxygenated blood from the lungs and pump it to the organs of the body to provide the organs with their metabolic needs for oxygen. The right portions of the heart draw deoxygenated blood from the body organs and pump it to the lungs where the blood gets oxygenated. These pumping functions result from contractions of the myocardium (cardiac muscles). In a normal heart, the sinoatrial node, the heart's natural pacemaker, generates electrical impulses, called action potentials, that propagate through an electrical conduction system to various regions of the heart to excite the myocardial tissues of these regions. Coordinated delays in the propagations of the action potentials in a normal electrical conduction system cause the various portions of the heart to contract in synchrony to result in efficient pumping functions. A blocked or otherwise abnormal electrical conduction and/or deteriorated myocardial tissue cause dyssynchronous contraction of the heart, resulting in poor hemodynamic performance including a diminished blood supply to the heart and the rest of the body. The condition in which the heart fails to pump enough blood to meet the body's metabolic needs is known as heart failure.

Myocardial infarction (MI) is the necrosis of portions of the myocardial tissue resulted from cardiac ischemia, a condition in which the myocardium is deprived of adequate oxygen supply and metabolite removal due to an interruption in blood supply caused by an occlusion of a blood vessel such as a coronary artery. The necrotic tissue, known as infarcted tissue, loses the contractile properties of the normal, healthy myocardial tissue. Consequently, the overall contractility of the myocardium is weakened, resulting in an impaired hemodynamic performance. Following an MI, cardiac remodeling starts with expansion of the region of infarcted tissue and progresses to a chronic, global expansion in the size and change in the shape of the entire left ventricle. The consequences include a further impaired hemodynamic performance and a significantly increased risk of developing heart failure.

When a blood vessel such as the coronary artery is partially or completely occluded, a revascularization procedure such as pharmacological reperfusion or mechanical reperfusion (percutaneous coronary intervention) can be performed to reopen the occluded blood vessel. In addition to the ischemic injury resulting from MI and percutaneous coronary intervention, reperfusion that follows the reopening of the occluded blood vessel is also known to cause cardiac injury, known as reperfusion injury. In addition, plaques dislodged and displaced by the revascularization procedure may enter small blood vessels branching from the blood vessel in which the revascularization is performed, causing occlusion of these small blood vessels. The revascularization procedure may also cause distal embolization, i.e., obstruction of the artery caused by the plaque dislodged during the procedure.

Thus, cardiac injury may result from both MI and its treatment. At least for these reasons, there is a need for minimizing cardiac injury associated with ischemia and reperfusion.

### SUMMARY

An external cardiac stimulation patch integrates a transcutaneous cardiac stimulation device and body-surface electrodes with a skin patch. The skin patch is to be attached onto a patient to provide for electrical contacts between the body-surface electrodes and a patient. The transcutaneous cardiac stimulation device delivers pacing pulses to the heart of the patient through pacing electrodes selected from the body-surface electrodes.

In one embodiment, a pacing system includes a transcutaneous cardiac stimulation device and a plurality of body-surface electrodes integrated with a skin patch. The transcutaneous cardiac stimulation device includes a pacing output circuit that produces pacing pulses suitable for capturing a patient's heart by transcutaneous delivery and a pacing control circuit that controls the transcutaneous delivery of the pacing pulses. The plurality of body-surface electrodes includes a pacing electrode set electrically wired to the pacing output circuit. The pacing electrode set includes at least two electrodes through which the pacing pulses are transcutaneously delivered to the heart. The skin patch is to be attached onto the patient's skin such that electrical contacts between the pacing electrode set and the patient's body allow for effective transcutaneous delivery of the pacing pulses to the heart.

In one embodiment, a method for pacing a heart is provided. Pacing pulses are transcutaneously delivered to a patient's heart from a transcutaneous cardiac stimulation device through a pacing electrode set. The pacing electrode set includes two or more electrodes selected from a plurality of body-surface electrodes. The transcutaneous cardiac stimulation device and the plurality of body-surface electrodes are integrated with a skin patch. The skin patch is attached onto the patient's skin such that electrical contacts between the pacing electrode set and the patient's body allow for effective transcutaneous delivery of the pacing pulses to the heart.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 is an illustration of an embodiment of an external cardiac stimulation system and portions of an environment in which the system is used.

FIG. 2 is a front-view illustration of an embodiment of an external cardiac stimulation patch of the external cardiac stimulation system.

FIG. 3 is a side, cross-sectional-view illustration of an embodiment of the external cardiac stimulation patch.

FIG. 4 is a rear-view illustration of an embodiment the external cardiac stimulation patch.

FIG. 5 is a block diagram illustrating an embodiment of a circuit of the cardiac stimulation patch.

FIG. 6 is a block diagram illustrating another embodiment of the circuit of the external cardiac stimulation patch.

FIG. 7 is a timing diagram illustrating an embodiment of a cardioprotective pacing protocol.

FIG. 8 is a block diagram illustrating another embodiment of the circuit of the external cardiac stimulation patch.

FIG. 9 is a block diagram illustrating an embodiment of a monitoring circuit of the external cardiac stimulation patch.

FIG. 10 is a block diagram illustrating an embodiment of a pacing control circuit of the external cardiac stimulation patch.

FIG. 11 is a block diagram illustrating an embodiment of a user interface of the external cardiac stimulation patch.

FIG. 12 is a flow chart illustrating an embodiment of a method for transcutaneously delivering cardiac pacing using the external cardiac stimulation patch.

FIG. 13 is a flow chart illustrating another embodiment of the method for transcutaneously delivering cardiac pacing using the external cardiac stimulation patch.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description provides examples, and the scope of the present invention is defined by the appended claims and their legal equivalents.

It should be noted that references to “an”, “one”, or “various” embodiments in this disclosure are not necessarily to the same embodiment, and such references contemplate more than one embodiment.

This document discusses, among other things, an external cardiac stimulation patch that integrates a cardiac stimulation device and electrodes with a skin patch. The skin patch is to be attached to the skin of a patient such that the electrodes securely contact the skin to allow for effective transcutaneous delivery of cardiac pacing pulses to capture the patient's heart. In various embodiments, the skin patch is attached to the skin using adhesive, belt, and/or other means that ensure secure contacts between the electrodes and the skin In one embodiment, the transcutaneous cardiac stimulation device also includes a defibrillator to transcutaneously deliver defibrillation shock pulses to the patient's heart.

In one embodiment, the external cardiac stimulation patch is used to treat a patient whose myocardium suffers ischemic injury from cardiac ischemia or MI. When available and appropriate, a revascularization procedure using pharmacological or mechanical means is performed to reopen the completely or partially occluded blood vessel associated with the ischemia or MI. An example of the pharmacological means is intravenous administration of tissue plasminogen activator (tPA), which dissolves the plaque that occludes the blood vessel. An example of the mechanical means is a percutaneous transluminal vascular intervention (PTVI) procedure, such as a percutaneous transluminal coronary angioplasty (PTCA) procedure. Such revascularization procedures may cause reperfusion injury when the blood vessel is reopened. The external cardiac stimulation patch transcutaneously delivers an acute pacing cardioprotection therapy to the patient according to a cardioprotective pacing protocol that specifies a pacing sequence for augmenting the patient's cardiac stress to a level effecting cardioprotection against the ischemic and reperfusion injuries. The use of such an external device allows the acute pacing cardioprotection therapy to be delivered promptly and non-invasively in response to a detection of the cardiac ischemia or MI, before, during, and/or after the revascularization procedure. In one embodiment, the external cardiac stimulation patch allows for the delivery of the acute pacing cardioprotection therapy before that patient reaches a hospital or other medical facilities where an invasive procedure can be performed, to avoid delay that would expand the extent of myocardial tissue damage resulting from ischemia and/or reperfusion. In one embodiment, the external cardiac stimulation patch is configured to be a consumer device that requires minimal training to operate, in a way similar to an automatic external defibrillator (AED).

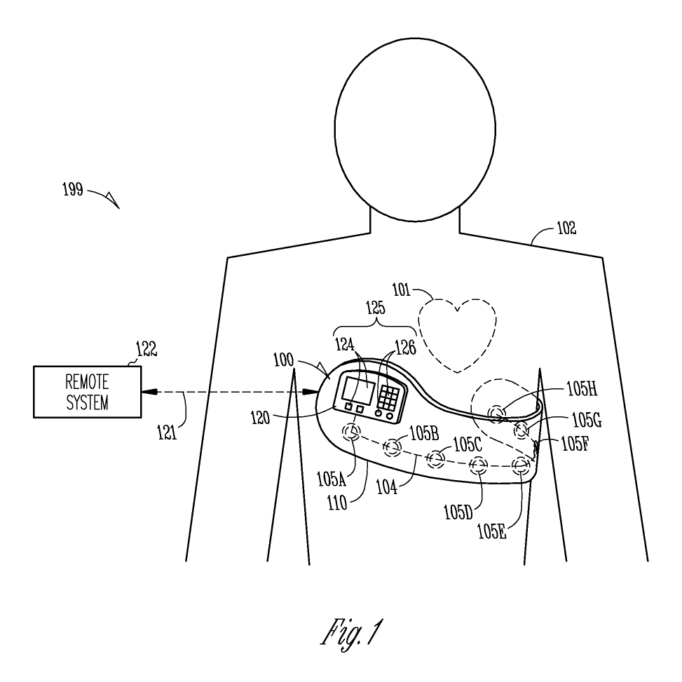

FIG. 1 is an illustration of an embodiment of an external cardiac stimulation system 199 and portions of an environment in which system 199 is used. System 199 includes an external cardiac stimulation patch 100, a remote system 122, and a telemetry link 121 providing for communication between external cardiac stimulation patch 100 and remote system 122.

External cardiac stimulation patch 100 is formed by integrating a transcutaneous cardiac stimulation device 120, body-surface electrodes 105A-H, and electrical conductors 104 with a skin patch 110. Conductors 104 electrically connect body-surface electrodes 105A-H to transcutaneous cardiac stimulation device 120. In various embodiments, external cardiac stimulation patch 100 is applied to a patient who is in need for cardiac stimulation, such as in response to an acute MI, or applied before an anticipated cardiac ischemia or reperfusion event, such as a revascularization procedure. As illustrated in FIG. 1, external cardiac stimulation patch 100 is to be attached to a body 102 to deliver cardiac stimulation to a heart 101.

Transcutaneous cardiac stimulation device 120 includes circuitry performing pacing, defibrillation, and/or monitoring functions and a user interface 125. User interface 125 allows a user to control the delivery of the cardiac stimulation. In the illustrated embodiment, user interface 125 includes user input devices 126 and presentation devices 124. User input devices 126 include devices such as buttons, keys, and knobs to receive commands from the user for starting, stopping, and adjusting the delivery of the cardiac stimulation such as pacing and defibrillation. Presentation devices 124 include devices such as light-emitting diodes (LEDs), liquid crystal display (LCD) and audio signal generator to indicate device status (such as battery status and on/off status), sensed events and parameters (such as heart beats and heart rate), and therapy delivery events and parameters (such as pacing pulse delivery and/or capture, pacing energy parameters, and pacing protocol being executed).

Body-surface electrodes 105A-H provide for electrical connections between transcutaneous cardiac stimulation device 120 and body 102 for transcutaneously delivering the cardiac stimulation to heart 101 while external cardiac stimulation patch 100 is attached onto body 102. In one embodiment, body-surface electrodes 105A-H also allow for sensing of surface biopotential signals such as surface electrocardiographic (ECG) signals. Body-surface electrodes 105A-H are shown in FIG. 1 for illustrative purposes only. In various embodiments, the number, shape, and locations of body-surface electrodes integrated with skin patch 110 depend on the need for the cardiac stimulation such as pacing and defibrillation and/or physiological monitoring such as ECG sensing. Body-surface electrodes 105A-H represent two or more body-surface electrodes in various embodiments. In one embodiment, body-surface electrodes 105A-H include two or more electrodes for pacing. In a further embodiment, the two or more electrodes also allow for defibrillation and/or sensing. In one embodiment, body-surface electrodes 105A-H include at least two electrodes for positioning on body 102 to form a transthoracic pathway for the pacing and/or defibrillation current. In one embodiment, body-surface electrodes 105A-H include three or more electrodes for selection based on pacing energy requirement and location of ischemia in heart 101. In one embodiment, body-surface electrodes 105A-H include a pacing electrode set including a plurality of electrodes selectable for pacing, a defibrillation electrode set including a plurality of electrodes selectable for defibrillation, and a monitoring electrode set including a plurality of electrodes selectable for physiological signal sensing such as ECG sensing. In a further embodiment, body-surface electrodes 105A-H include one or more common electrodes shared by two or more of the pacing electrode set, defibrillation electrode set, and monitoring electrode set.

In another embodiment, one or more electrodes of body-surface electrodes 105A-H are replaced by one or more needle electrodes for better electrical conductivity. When transcutaneous cardiac stimulation device 120 is unable to capture heart 101 by transcutaneously delivery pacing pulses, the one or more needle electrodes may lower pacing energy required to capture heart 101, thereby allowing for an effective pacing therapy.

Skin patch 110 has an attachment surface that includes a substantial portion in contact with the skin of body 102 when the cardiac stimulation is delivered. In one embodiment, body-surface electrodes 105A-H are affixed onto the attachment surface. Skin patch 110 is to be attached onto the skin of body 102 such that electrical contacts between body-surface electrodes 105A-H and body 102 allow for effective transcutaneous delivery of cardiac stimulation to heart 101. In various embodiments, skin patch 110 is to be attached onto body 102 using affixation means configured to secure the electrical contacts by preventing displacement of body-surface electrodes 105A-H relative to body 102 during operation of external cardiac stimulation patch 100. In one embodiment, the affixation means includes an adhesive layer on the attachment surface of skin patch 110. In another embodiment, the affixation means includes one or more belts/straps and means for pressing the body-surface electrodes against the skin, such as springs or sponge-like structure behind each electrode.

Remote system 122 communicates with transcutaneous cardiac stimulation device 120. In one embodiment, remote system 122 communicates with transcutaneous cardiac stimulation device 120 wirelessly via telemetry link 121. In one embodiment, remote system 122 includes a screen to display signals and parameters sensed by external cardiac stimulation patch 100. In one embodiment, body-surface electrodes 105A-H represent an electrode set allowing for sensing of the standard 12-lead ECG, and the screen displays the sensed 12-lead ECG.

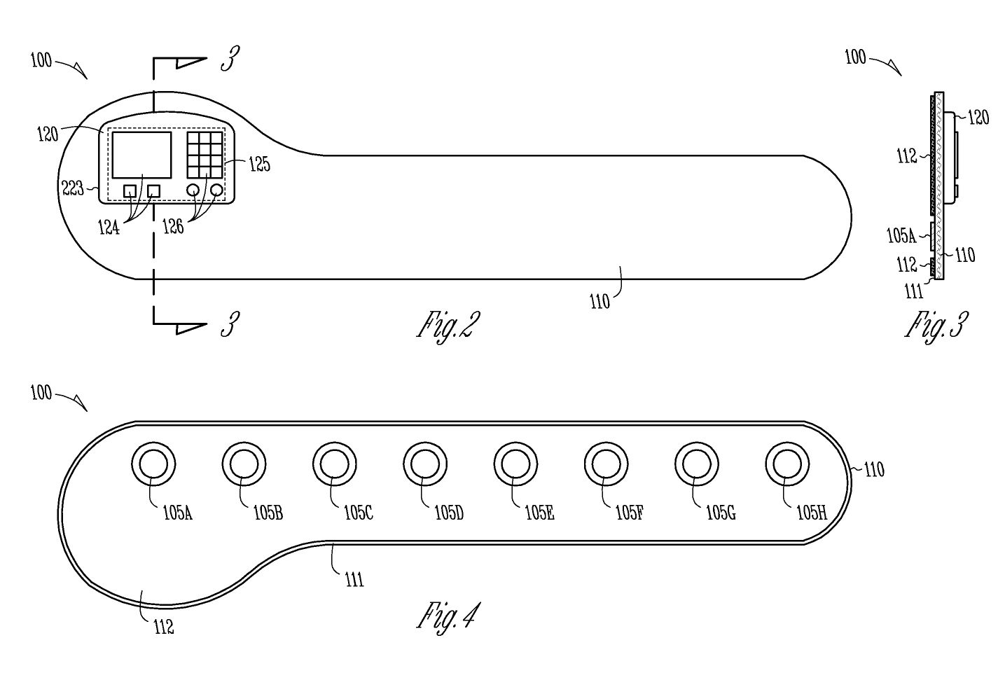

FIG. 2 is a front-view illustration of an embodiment of external cardiac stimulation patch 100. The front-view illustration shows the front side of external cardiac stimulation patch 100, which faces outside (away from body 102) when external cardiac stimulation patch 100 is attached onto body 102. In the illustrated embodiment, transcutaneous cardiac stimulation device 120 is incorporated onto the front side. In one embodiment, transcutaneous cardiac stimulation device 120 includes a housing 223 containing electronic circuitry and user interface 125. In one embodiment, housing 223 is flexible. In a further embodiment, the electronic circuitry is constructed on a flexible substrate, such that the entire external cardiac stimulation patch 100 is substantially flexible. In one embodiment, the electronic circuitry is capable of delivering a pacing cardioprotection therapy that protects heart 101 from the ischemic and reperfusion injuries. User interface 125, including presentation devices 124 and user input devices 126, is incorporated onto housing 223 to allow a user to control the delivery of the pacing cardioprotection therapy.

FIG. 3 is a side, cross-sectional-view illustration of an embodiment of external cardiac stimulation patch 100 (except that transcutaneous cardiac stimulation device 120 is not shown as a cross-sectional view). The side, cross-sectional-view illustration shows an attachment surface 111 and an adhesive layer 112. Attachment surface 111 includes a substantial portion in contact with the skin of body 102 when the cardiac stimulation is delivered. Adhesive layer 112 covers a substantial portion of attachment surface 111.

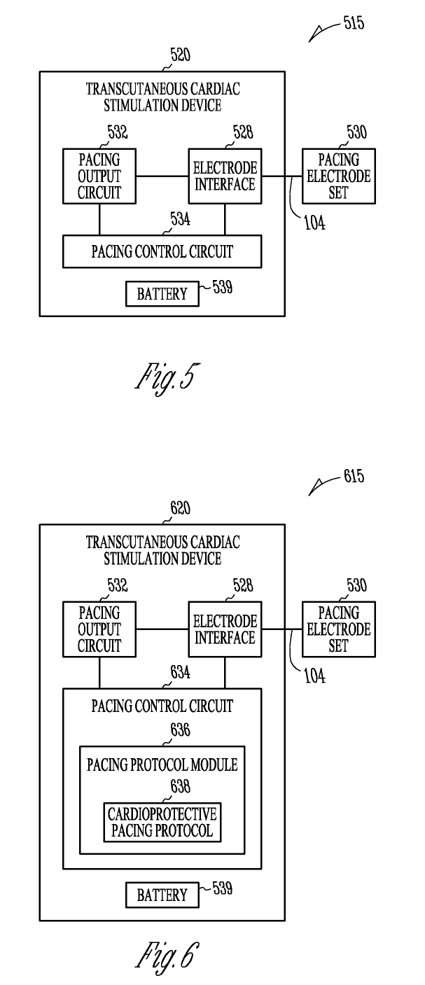

FIG. 4 is a rear-view illustration of an embodiment of external cardiac stimulation patch 100. The rear-view illustration shows the rare side of external cardiac stimulation patch 100, which faces body 102 when external cardiac stimulation patch 100 is attached onto body 102. In one embodiment, body-surface electrodes 105A-H are each a disk electrode affixed onto the rear side. In various embodiments, adhesive layer 112 covers a substantial portion of attachment surface 111. In the illustrated embodiment, adhesive layer 112 covers approximately the entire attachment surface 111 except for the areas where body-surface electrodes 105A-H are exposed for electrical connections to the skin of body 102.

In one embodiment, transcutaneous cardiac stimulation device 120 delivers pacing cardioprotection therapies including pacing pre-conditioning (cardioprotective pacing before the onset of an anticipated ischemic or reperfusion event) and pacing post-conditioning (cardioprotective pacing after the onset of the ischemic or reperfusion event). In one embodiment, transcutaneous cardiac stimulation device 120 also delivers defibrillation pulses through a defibrillation electrode set including two or more electrodes selected from body-surface electrodes 105A-H. In one embodiment, transcutaneous cardiac stimulation device 120 includes an automatic external defibrillator (AED) with pacing capability. Thus, external cardiac stimulation patch 100 is capable of providing for non-invasive, transthoracic, and transcutaneous delivery of pacing cardioprotection and defibrillation therapies.

In one embodiment, external cardiac stimulation patch 100 is used to deliver the pacing cardioprotection therapy when a percutaneous or implantable stimulation system is not timely available or not suitable for the patient. For example, if prompt delivery of the pacing cardioprotection therapy is beneficial to the patient, external cardiac stimulation patch 100 is used when the patient is in an ambulance, or in an emergency room or catheterization laboratory waiting for a revascularization procedure.

In various embodiments, external cardiac stimulation patch 100 includes any number and configuration of body-surface electrodes suitable for delivering the pacing pulses and performing other functions discussed in this document. In various embodiments, the circuit of transcutaneous cardiac stimulation device 120, including its various elements discussed in this document, is implemented using a combination of hardware and software. In various embodiments, each element of transcutaneous cardiac stimulation device 120 discussed in this document may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

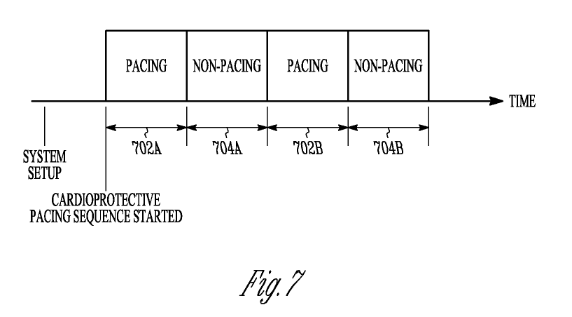

FIG. 5 is a block diagram illustrating an embodiment of a circuit 515 of external cardiac stimulation patch 100. Circuit 515 includes a transcutaneous cardiac stimulation device 520 electrically wired to a pacing electrode set 530 via conductors 104. Transcutaneous cardiac stimulation device 520 is an embodiment of transcutaneous cardiac stimulation device 120 and includes a pacing output circuit 532, a pacing control circuit 534, an electrode interface 528, and a battery 539. Pacing output circuit 532 produces pacing pulses suitable for capturing heart 101 by transcutaneous delivery through pacing electrode set 530. Pacing control circuit 534 controls the transcutaneous delivery of the pacing pulses. Electrode interface 528 provides for routing between pacing output circuit 532 and pacing electrode set 530. Battery 530 provides energy for the operation of circuit 515. Pacing electrode set 530 includes two or more electrodes attached onto body 102. In one embodiment, pacing electrode set 530 includes two or more electrodes selected from a plurality of body-surface electrodes such as electrodes 105A-H. In one embodiment, the selection of pacing electrode set 530 from the plurality of body-surface electrodes is adjustable by programming electrode interface 528.

FIG. 6 is a block diagram illustrating an embodiment of a circuit 615 of external cardiac stimulation patch 100. Circuit 615 is an embodiment of circuit 515 and includes a transcutaneous cardiac stimulation device 620 electrically wired to pacing electrode set 530. Transcutaneous cardiac stimulation device 620 is an embodiment of transcutaneous cardiac stimulation device 520 and includes pacing output circuit 532, a pacing control circuit 634, electrode interface 528, and battery 539. Pacing control circuit 634 includes a pacing protocol module 636 and controls the transcutaneous delivery of the pacing pulses by executing a pacing protocol. Pacing protocol module 636 stores one or more pacing protocols according to which the pacing pulses are delivered. In the illustrated embodiment, pacing protocol module 636 stores one or more pacing protocols including a cardioprotective pacing protocol 638. Cardioprotective pacing protocol 638 specifies a pacing sequence according to which pacing pulses are delivered to augment mechanical stress on the myocardium of heart 101 to a level effecting cardioprotection against myocardial injury. Battery 539 provides energy for the operation of circuit 615.

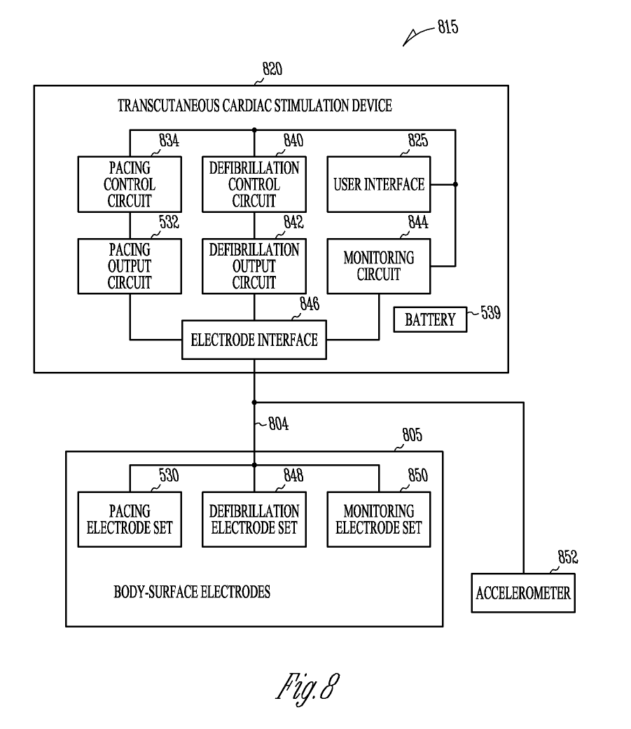

FIG. 7 is a timing diagram illustrating an embodiment of cardioprotective pacing protocol 638, which specifies a cardioprotective pacing sequence. The cardioprotective pacing sequence is started after a pacing system such as system 100 is set up as illustrated in FIG. 1. In various embodiments, the cardioprotective pacing sequence is started before, during, and/or after an ischemic or reperfusion event. Such a cardioprotective pacing sequence is delivered to reduce the extent of myocardial damage resulting from ischemia and/or reperfusion injury;

As illustrated in FIG. 7, the cardioprotective pacing sequence includes alternating pacing and non-pacing periods. Each pacing period is a pacing duration during which the pacing pulses are delivered in a specified pacing mode. Each non-pacing period is a non-pacing duration during which no pacing pulse is delivered. FIG. 7 shows, by way of example, a cardioprotective pacing sequence that includes two cycles of alternating pacing and non-pacing periods: pacing period 702A, non-pacing periods 704A, pacing period 702B, and non-pacing periods 704B. In one embodiment, the number of the cycles of alternating pacing and non-pacing periods is programmable, and each of the pacing and non-pacing periods is programmable. In the illustrated embodiments, the cardioprotective pacing sequence starts with a pacing period. In another embodiment, the cardioprotective pacing sequence starts with a non-pacing period.

In one embodiment, the cardioprotective pacing sequence is initiated before the ischemic or reperfusion event and includes approximately 1 to 10 cycles of alternating pacing and non-pacing periods. The pacing period is in a range of approximately 15 seconds to 20 minutes. The non-pacing period is in a range of approximately 5 seconds to 20 minutes. In a specific example, the cardioprotective pacing sequence initiated before the ischemic or reperfusion event includes 3 cycles of alternating pacing and non-pacing periods each being approximately 5-minute long. In one embodiment, the cardioprotective pacing sequence is initiated during the ischemic or reperfusion event and includes approximately 1 to 10 cycles of alternating pacing and non-pacing periods. The pacing period is in a range of approximately 15 seconds to 20 minutes. The non-pacing period is in a range of approximately 5 seconds to 20 minutes. In a specific example, the cardioprotective pacing sequence delivered during the ischemic or reperfusion event includes 3 cycles of alternating pacing and non-pacing periods each being approximately 5-minute long. In one embodiment, the cardioprotective pacing sequence is initiated after the ischemic or reperfusion event and includes approximately 1 to 10 cycles of alternating pacing and non-pacing periods. The pacing period is in a range of approximately 10 seconds to one minute. The non-pacing period is in a range of approximately 5 seconds to one minute. In one specific example, the cardioprotective pacing sequence delivered after the ischemic or reperfusion event includes 2 to 4 cycles of alternating pacing and non-pacing periods each being approximately 30-second long.

The specified pacing mode is selected to augment the mechanical stress on the patient's myocardium to a level effecting cardioprotection against myocardial injury by delivering the pacing pulses. In one embodiment, during each pacing period, rapid, asynchronous pacing under VOO or AOO mode is applied. In other words, pacing pulses are delivered at a rate substantially higher than the patient's intrinsic heart rate without being synchronized to the patient's intrinsic cardiac contractions. The pacing rate is set to, for example, 10-20 beats per minute above the patient's intrinsic heart rate. In various other embodiments, the cardioprotective pacing sequence includes pacing at one or more atrial tracking or other pacing modes, depending on the availability of reliable atrial and/or ventricular sensing using electrodes selected from body-surface electrodes 105A-H. Reliable atrial sensing allows for reliable detection of atrial depolarizations (P waves). Reliable ventricular sensing allows for reliable detection of ventricular depolarizations (R waves). Examples of such pacing modes include the VVI, AAI, VDD, and DDD modes. VVI mode pacing may be applied during each pacing period when reliable ventricular sensing is available, with the lower rate limit set to a value substantially higher than the patient's intrinsic ventricular rate, such as by 10-20 beats per minute. AAI mode pacing may be applied during each pacing period when reliable atrial sensing is available, with the lower rate limit set to a value substantially higher than the patient's intrinsic atrial rate, such as by 10-20 beats per minute. VDD mode pacing may be applied during each pacing period when reliable atrial sensing and ventricular sensing are available, with the atrioventricular (AV) delay set to a value that is substantially shorter than the patient's intrinsic AV interval. DDD mode pacing may also be applied during each pacing period when reliable atrial sensing and ventricular sensing are available, with the lower rate limit set to a value substantially higher than the patient's intrinsic heart rate, and/or the AV delay set to a value that is substantially shorter than the patient's intrinsic AV interval.

In one embodiment, prior to the delivery of the cardioprotective pacing sequence and/or during each of the non-pacing periods, transcutaneous cardiac stimulation device 620 operates in a sensing-only mode during which the patent's intrinsic heart rate and/or AV interval are measured. Examples of the sensing-only modes include OVO, OAO, and ODO modes, the selection of which depending on whether atrial sensing, ventricular sensing, or both are required by the specified pacing mode. In one embodiment, the delivery of the pacing pulses during each of the pacing periods is paused for one or more brief periods of sensing-only mode to allow for measurement of the patient's intrinsic heart rate and/or AV delay in order to adjust the pacing rate when needed. For example, the delivery of the pacing pulses is paused repeatedly for about 5 seconds for each minute of the pacing period.

FIG. 8 is a block diagram illustrating an embodiment of a circuit 815 of external cardiac stimulation patch 100. Circuit 815 is another embodiment of circuit 515 and includes a transcutaneous cardiac stimulation device 820, body-surface electrodes 805, an accelerometer 852, and conductors 804 through which electrodes 805 and accelerometer 852 are wired to device 820.

Transcutaneous cardiac stimulation device 820 is an embodiment of transcutaneous cardiac stimulation device 520 and includes pacing output circuit 532, a defibrillation output circuit 842, a monitoring circuit 844, a pacing control circuit 834, a defibrillation control circuit 840, a user interface 825, an electrode interface 846, and battery 539. Defibrillation output circuit 842 delivers defibrillation pulses suitable for defibrillating the heart by transcutaneous delivery. Monitoring circuit 844 monitors the patient's conditions related to the delivery of the pacing pulses and defibrillation pulses. Pacing control circuit 834 performs the functions of pacing control circuit 534 and in addition, controls the delivery of the pacing pulses using the patient's conditions monitored by monitoring circuit 844 and coordinates the delivery of the pacing pulses with the delivery of the defibrillation pulses. Defibrillation control circuit 840 controls the delivery of the defibrillation pulses. In one embodiment, defibrillation control circuit 840 initiates the delivery of a defibrillation pulse in response to one of a defibrillation command received by user interface 825 and a tachyarrhythmia detection signal indicative of the detection of a tachyarrhythmia episode by monitoring circuit 844. The tachyarrhythmia episode to be detected includes one or more specified types indicated for a defibrillation therapy. Electrode interface 846 provides for routing between transcutaneous cardiac stimulation device 820 and body-surface electrodes 805. In one embodiment, electrode interface 846 includes programmable connections between transcutaneous cardiac stimulation device 820 and body-surface electrodes 805 for routing the pacing pulses, defibrillation pulses, and signals indicative the patient's conditions being monitored. User interface 825 is an embodiment of user interface 125 and receives user commands for controlling the delivery of pacing and defibrillation pulses and presents various signals indicative of the patient's conditions and/or operational status of transcutaneous cardiac stimulation device 820. Battery 539 provides circuit 815 with energy for its operation.

Body-surface electrodes 805 are configured for skin attachment and include a pacing electrode set 530, a defibrillation electrode set 848, and a monitoring electrode set 850. One example of body-surface electrodes 805 includes electrodes 105A-H. Defibrillation output circuit 842 delivers the defibrillation pulses transcutaneously through defibrillation electrode set 848, which includes two or more electrodes selected from body-surface electrodes 805. In one embodiment, monitoring electrode set 850 includes two or more surface ECG electrodes allowing for sensing of ECG signals. In a specific embodiment, monitoring electrode set 850 allows for sensing the standard 12-lead ECG.

Pacing electrode set 530, defibrillation electrode set 848, and monitoring electrode set 850 are each selected from body-surface electrodes 805. In various embodiments, each electrode of body-surface electrodes 805 is selectable for being an electrode of one, two, or all of pacing electrode set 530, defibrillation electrode set 848, and monitoring electrode set 850. In various embodiments, two or all of pacing electrode set 530, defibrillation electrode set 848, and monitoring electrode set 850 share one or more common electrodes.

In the illustrated embodiment, accelerometer 852 is integrated with skin patch 110, in addition to transcutaneous cardiac stimulation device 820 and body-surface electrodes 805. Accelerometer 852 senses an acceleration signal indicative of skeletal muscle contractions resulting from the pacing pulses delivered through pacing electrode set 530.

FIG. 9 is a block diagram illustrating an embodiment of a monitoring circuit 944, which is an embodiment of monitoring circuit 844. In the illustrated embodiment, monitoring circuit 944 includes an ECG amplifier circuit 952, a capture verification circuit 954, an ischemia detection circuit 956, a tachyarrhythmia detection circuit 958, and a hemodynamic sensing circuit 959. ECG amplifier circuit 952 senses one or more ECG signals through monitoring electrode set 850 and processes the one or more ECG signals. In one embodiment, ECG amplifier circuit 952 senses and processes the standard 12-lead ECG.

Capture verification circuit 954 determines whether each pacing pulse delivered through pacing electrode set 530 results in a cardiac depolarization using the one or more ECG signals. In one embodiment, capture verification circuit 954 further determines a capture percentage associated with pacing electrode set 530 and one or more specified pacing energy parameters including pacing amplitude and/or pulse width. The capture percentage is a percentage of the pacing pulses resulting in cardiac depolarizations. Capture verification circuit 954 produces a capture verification signal indicative of the capture percentage.

Ischemia detection circuit 956 detects an ischemic event, such as an acute MI, using the one or more ECG signals and produces an ischemia signal indicative of a detection of the ischemic event. In one embodiment, ischemia detection circuit 956 detects the ischemic event by detecting an elevated level of S-T segment amplitude on at least one ECG signal sensed by ECG amplifier circuit 952. Ischemia detection circuit 956 indicates an occurrence of ischemia when the elevated level of S-T segment amplitude is detected, for example, when the S-T segment amplitude exceeds an ischemia threshold. In one embodiment, ischemia detection circuit 956 also indicates an onset of reperfusion when the S-T segment amplitude falls from the elevated level to a level below the ischemia threshold. In one embodiment, ischemia detection circuit 956 locates an ischemic region in heart 101 using ECG signals sensed by ECG amplifier circuit 952 and produces an ischemia signal indicative of an approximate location of the ischemic region. The ischemic region is located using S-T segment amplitudes on ECG signals sensed through multiple electrode pairs of monitoring electrode set 850. In one embodiment, pacing control circuit 834 selects pacing electrode set 530 from body-surface electrodes 805 based on the approximate location of the ischemic region. Depending on the purpose of pacing, pacing electrode set 530 is selected, for example, for directing the pacing pulses to ischemic or non-ischemic regions of heart 101. In one embodiment, pacing electrode set 530 is selected for avoiding stressing the ischemic region. In another embodiment, pacing electrode set 530 is selected for reducing stress on the ischemic region while augmenting stress on one or more non-ischemic regions.

Tachyarrhythmia detection circuit 958 detects one or more specified types of tachyarrhythmia indicated for defibrillation therapy using the one or more ECG signals and produces a tachyarrhythmia detection signal indicative of a detection of a specified type tachyarrhythmia. In one embodiment, defibrillation control circuit 840 initiates the delivery of a defibrillation pulse in response to the tachyarrhythmia detection signal. This allows timely treatment of tachyarrhythmia that occurs during the delivery of the pacing cardioprotection therapy.

Hemodynamic sensing circuit 959 senses a signal indicative of hemodynamic performance of the patient to allow for titration of the pacing cardioprotection therapy. In one embodiment, hemodynamic sensing circuit 959 senses an impedance signal using electrodes selected from body-surface electrodes 105A-H. Pacing control circuit 834 then measures stroke impedance from the impedance signal and adjust one or more pacing parameters using the stroke impedance. An increase in the stroke impedance indicates an improvement in hemodynamic performance. In one embodiment, to sense the impedance signal, hemodynamic sensing circuit 959 injects an electrical current into body 102 through an electrode in an anterior position (e.g., electrode 105A) and another electrode in a posterior position (e.g., electrode 105H). Then, hemodynamic sensing circuit 959 senses a voltage through the same electrodes (e.g., electrodes 105A and 105H) or adjacent electrodes (e.g., electrodes 105B and 105G). The impedance equals the sensed voltage divided by the injected electrical current.

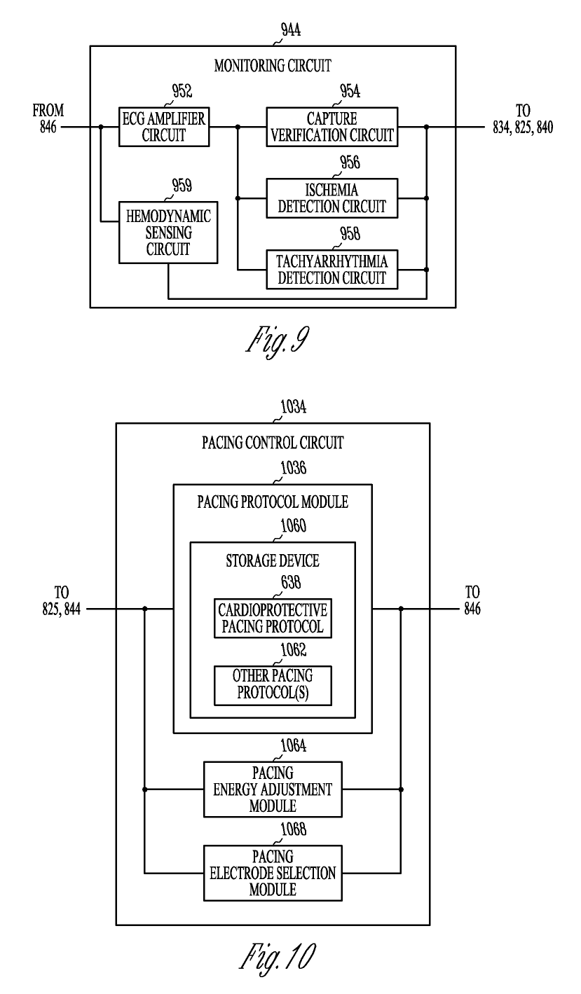

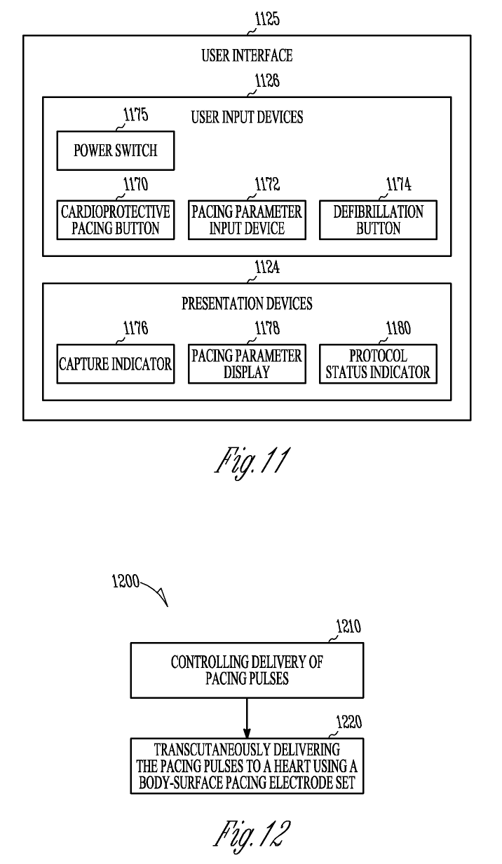

FIG. 10 is a block diagram illustrating an embodiment of a pacing control circuit 1034, which is an embodiment of pacing control circuit 834. Pacing control circuit 1034 includes a pacing protocol module 1036, a pacing energy adjustment module 1064, and a pacing electrode selection module 1068. In one embodiment, pacing control circuit 1034 starts executing a pacing protocol in response to a pacing command received from the user by user interface 825. In one embodiment, pacing control circuit 1034 stops executing the pacing protocol in response to one of a pacing termination command received from the user by user interface 825 and the tachyarrhythmia detection signal produced by tachyarrhythmia detection circuit 958. The pacing protocol includes cardioprotective pacing protocol 638.

Pacing protocol module 1036 includes a storage device 1060 that stores at least cardioprotective pacing protocol 638. In the illustrated embodiment, storage device 1060 also stores one or more other pacing protocols 1062, such as an anti-arrhythmia pacing protocol specifying a pacing therapy to be delivered in coordination with the defibrillation therapy. In one embodiment, cardioprotective pacing protocol 638 is a patient-specific protocol customized for each individual patient, with pacing parameters selected and/or adjusted based on the patient's current conditions and cardiac history. Examples of such pacing parameters include the number of pacing cycles (each including a pacing period and a non-pacing period), the pacing period, the non-pacing period, the pacing energy parameter(s) including pacing amplitude and/or pulse width, and the pacing mode. In one embodiment, cardioprotective pacing protocol 638 is a disease-specific protocol customized for one or more cardiac and/or non-cardiac diseases. Certain diseases affect the effectiveness of the pacing cardioprotection therapy and hence require higher dosage of the pacing cardioprotection therapy. For example, higher pacing energy is likely required for producing the cardioprotective effect when the patient is diabetic.

Pacing energy adjustment module 1064 determines the pacing energy parameters associated with pacing electrode set 530. The pacing energy parameters include pacing amplitude and pulse width. At least one of the pacing amplitude and pulse width is adjustable. In one embodiment, the pacing pulses are of constant-current-type with the pacing amplitude adjustable in a range between approximately 10 to 140 milliamperes, and the pacing pulse width adjustable in a range between approximately 2 to 100 milliseconds. In one embodiment, pacing energy adjustment module 1064 adjusts one or more of the pacing energy parameters by comparing the capture percentage to a capture threshold. The capture threshold is specified to a satisfactory or acceptable value, such as 80%. Pacing energy adjustment module 1064 increases pacing energy if the capture percentage is below the specified capture threshold. In one embodiment, pacing energy adjustment module 1064 receives a skeletal muscular stimulation signal indicative of a level of the patient's skeletal muscular contractions resulting from the pacing pulses, and adjusts the pacing energy using the capture percentage and the skeletal muscular stimulation signal. In one embodiment, the skeletal muscular stimulation signal is the acceleration signal sensed by accelerometer 852. Pacing energy adjustment module 1064 decreases the pacing energy if the acceleration signal is above a specified stimulation threshold while the capture percentage exceeds the specified capture threshold. In other embodiments, the skeletal muscular stimulation signal includes any signal indicative of the level of the skeletal muscular contractions, such as a displacement signal indicative of muscular movements or a biopotential signal indicative of myoelectric activities. In one embodiment, pacing energy adjustment module 1064 adjusts the pacing energy dynamically during the execution of cardioprotective pacing protocol 638. In one embodiment, pacing energy adjustment module 1064 adjusts one or more of the pacing energy parameters when the capture percentage is below the specified capture threshold. In another embodiment, pacing energy adjustment module 1064 adjusts one or more of the pacing energy parameters to maintain the capture percentage at about the specified capture threshold. In various embodiments, pacing energy adjustment module 1064 dynamically adjusts one or more of the pacing energy parameters during the execution of the cardioprotective pacing protocol to ensure effectiveness of the pacing cardioprotection therapy.

Pacing electrode selection module 1068 adjusts the selection of pacing electrode set 530 from body-surface electrodes 805 using one or more of the capture percentage, the pacing energy parameters, and the ischemia signal. For example, when the capture percentage falls below the specified capture threshold while the pacing energy cannot be increased, such as when the maximum energy capability of pacing output circuit 532 is reached or when the acceleration signal indicates an unacceptable level of skeletal muscular stimulation resulting from the pacing pulses, pacing electrode selection module 1068 selects a different pacing electrode set 530 from body-surface electrodes 805. In another example, pacing electrode selection module 1068 selects pacing electrode set 530 from body-surface electrodes 805 to avoid directing the pacing pulses to the ischemic region using the ischemia signal.

FIG. 11 is a block diagram illustrating an embodiment of a user interface 1125, which is an embodiment of user interface 825. User interface 1125 includes user input devices 1126 and presentation devices 1124.

User input devices 1126 include a power switch 1175, a cardioprotective pacing button 1170, a pacing parameter input device 1172, and a defibrillation button 1174. Power switch 1175 allows the user to turn transcutaneous cardiac stimulation device 820 on and off. Cardioprotective pacing button 1170 receives the cardioprotective pacing command from the user to start an execution of the cardioprotective pacing protocol. Pacing parameter input device 1172 allows adjustment of one or more programmable pacing parameters. In one embodiment, pacing parameter input device 1172 allows the user to overwrite parameter values specified in the stored cardioprotective pacing protocol and/or the parameter values produced by pacing energy adjustment module 1064. Defibrillation button 1174 receives the defibrillation command that initiates the delivery of a defibrillation pulse. The defibrillation command also stops the execution of the cardioprotective pacing protocol. In various embodiments, user input devices 1126 includes devices to receive other user commands, such as the pacing termination command that stops the execution of the cardioprotective pacing protocol. In various embodiments, functions of cardioprotective pacing button 1170, pacing parameter input device 1172, and defibrillation button 1174 are performed by user input devices in any configurations that are capable of receiving commands from the user, such as push buttons, keypad, dials, and interactive screen.

Presentation devices 1124 include a capture indicator 1176, a pacing parameter display 1178, and a protocol status indicator 1180. Capture indicator 1176 indicate whether each pacing pulse delivered through pacing electrode set 530 captures the heart. In one embodiment, capture indicator 1176 indicates the capture percentage. Pacing parameter display 1178 presents values of selected pacing parameters, including the parameters that are adjustable using pacing parameter input device 1172. Protocol status indicator 1180 indicates one or more of whether the cardioprotective pacing protocol is being executed, whether the pacing pulses are being delivered, and the percentage of the cardioprotective pacing sequence that has been completed. In various embodiments, functions of capture indicator 1176, pacing parameter display 1178, and protocol status indicator 1180 are performed by presentation devices in any configurations that are visible or otherwise perceivable by the user, such as a display screen and light-emitting diodes.

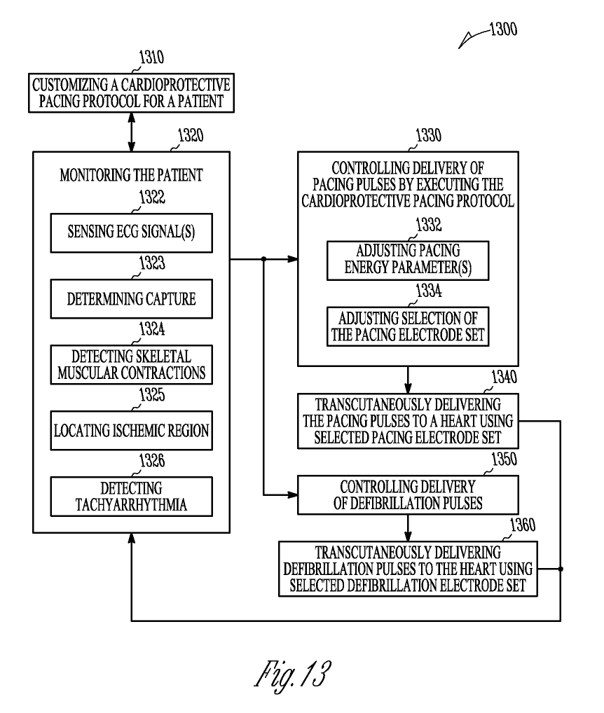

FIG. 12 is a flow chart illustrating an embodiment of a method 1200 for transcutaneously delivering cardiac pacing using the external cardiac stimulation patch. In one embodiment, method 1200 is performed by external cardiac stimulation patch 100, including its various embodiments discussed above with reference to FIGS. 1-11.

At 1210, delivery of pacing pulses is controlled. In one embodiment, the delivery of pacing pulses is controlled by automatically executing a pacing protocol. One example of such a pacing protocol includes a cardioprotective pacing protocol that specifies a cardioprotective pacing sequence following which pacing pulses are delivered to augment mechanical stress on the myocardium of a patient's heart to a level effecting cardioprotection against myocardial injury. An example of the cardioprotective pacing protocol is discussed with reference to FIG. 7. At 1220, the pacing pulses are delivered to the patient's heart from a transcutaneous cardiac stimulation device through a pacing electrode set. The pacing electrode set includes at least two electrodes selected from a plurality of body-surface electrodes. The body-surface electrodes and the transcutaneous cardiac stimulation device are electrically connected and integrated with a skin patch for attachment onto the patient.

FIG. 13 is a flow chart illustrating of an embodiment of a method 1300 for transcutaneously delivering cardioprotective pacing, which represents a specific embodiment of method 1200. In one embodiment, method 1300 is performed by external cardiac stimulation patch 100, including the various embodiments of circuit 815 discussed above with reference to FIGS. 8-11. To perform method 1300, external cardiac stimulation patch 100 is attached onto body 102 as illustrated in FIG. 1.

At 1310, the cardioprotective pacing protocol is customized for a patient. In one embodiment, the customization of the cardioprotective pacing protocol for the patient includes determination of values of one or more parameters based on the patient's medical history and examination results. Examples of such parameters include the number of pacing cycles (each including a pacing period and a non-pacing period), the pacing period, the non-pacing period, the pacing energy parameters such as the amplitude and pulse width, and pacing mode. These parameters determine the therapy dose, energy of each pacing pulse, and level of stress applied to the myocardium.

At 1320, the patient is monitored in preparation to and during the execution of the cardioprotective pacing protocol. In the illustrated embodiment, the patient monitoring includes sensing one or more ECG signals at 1322, determining capture at 1323, detecting skeletal muscular contractions at 1324, locating ischemic region at 1325, and detecting tachyarrhythmia at 1326. In one embodiment, the standard 12-lead ECG is sensed at 1322. In another embodiment, one or more ECG signals required for controlling the pacing according to the cardioprotective pacing protocol are sensed at 1322. Capture is verified at 1323 by determining whether each delivered pacing pulse results in a cardiac depolarization. In one embodiment, a capture percentage is determined at 1323. The capture percentage is the percentage of the pacing pulses resulting in cardiac depolarization and is associated with the pacing electrodes and the pacing energy parameters used. A capture verification signal indicative of the capture percentage is produced for the selected pacing electrode set and the specified pacing energy parameters. At 1324, skeletal muscular contractions caused by the pacing pulses are detected by sensing a skeletal muscular stimulation signal indicative of skeletal muscle contractions resulting from the pacing pulses. In one embodiment, an accelerometer senses an acceleration signal as the skeletal muscular stimulation signal. The capture percentage and the acceleration signal allow for adjustment of pacing energy to obtain the intended therapeutic effect of pacing while minimizing the patient's discomfort caused by the pacing-induced skeletal muscle contractions. At 1325, an ischemic region in the heart is approximately located using multiple ECG signals. This allows for directing the pacing pulses to target on, or to avoid targeting on the ischemic region. At 1326, tachyarrhythmia of one or more specified types is detected. The specified types are those requiring defibrillation therapy. The detection of the specified type tachyarrhythmia indicates a need to defibrillate the patient and/or a need to terminate or suspend the delivery of pacing pulses.

At 1330, the delivery of the pacing pulses is controlled by executing the cardioprotective pacing protocol. The execution is started in response to a pacing command received from a user. In one embodiment, the execution is stopped in response to a pacing termination command received from the user, or in response to a detection of the specified type tachyarrhythmia. Control of the delivery of the pacing pulses includes adjusting one or more of the pacing energy parameters at 1332 and adjusting selection of the pacing electrode set at 1334. One or both of the pacing amplitude and pacing pulse width are adjustable for controlling the pacing energy. The pacing electrode set is adjusted by selecting two or more electrodes from the plurality of available body-surface electrodes. In one embodiment, the parameter adjustment and/or electrode selection are performed manually by the user. In another embodiment, the parameter adjustment and/or electrode selection are performed automatically by a device, such as transcutaneous cardiac stimulation device 820. In one embodiment, the parameter adjustment and/or electrode selection are performed before the execution of the cardioprotective pacing protocol. In another embodiment, one or more of the pacing energy parameters are dynamically adjustable during the execution of the cardioprotective pacing protocol. In another embodiment, the selection of the pacing electrode set is also dynamically adjustable during the execution of the cardioprotective pacing protocol. At 1332, one or more of the pacing energy parameters are adjusted based on the detected capture percentage and the level of skeletal muscular contractions. In one embodiment, one or more of the pacing energy parameters are adjusted to maintain the pacing energy at approximately a specified capture threshold, such as about 80%, thereby securing the intended therapeutic effect of the cardioprotective pacing protocol while minimizing the patient's skeletal muscular stimulation. At 1334, the selection of the pacing electrode set is adjusted using one or more of the capture percentage, the one or more pacing energy parameters, and the location of the ischemic region. For example, two more electrodes are selected from the plurality of available body-surface electrodes to ensure that the capture percentage can be maintained at about the specified capture threshold, to use the lowest possible pacing energy, and/or to target or avoid the ischemic region. In one embodiment, one or more pacing parameters are adjusted at 1330 using a hemodynamic signal. One example of the hemodynamic signal is an impedance signal sensed using two more electrodes selected from the plurality of available body-surface electrodes. At 1340, the pacing pulses are transcutaneously delivered to the patient's heart using the selected pacing electrode set.

At 1350, delivery of defibrillation pulses is controlled. In one embodiment, one or more defibrillation pulses are delivered in response to one of a defibrillation command received from the user and the detection of the specified type tachyarrhythmia. At 1360, the one or more defibrillation pulses are transcutaneously delivered to the patient's heart using a defibrillation electrode set selected from the plurality of body-surface electrodes.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for pacing a heart having a myocardium in a living body having a skin, the system comprising:
a transcutaneous cardiac stimulation device including:
a pacing output circuit configured to produce pacing pulses suitable for capturing the heart by transcutaneous delivery; and
a pacing control circuit coupled to the pacing output circuit and configured to control the transcutaneous delivery of the pacing pulses;
a plurality of body-surface electrodes including a pacing electrode set electrically wired to the pacing output circuit, the pacing electrode set including at least two electrodes through which the pacing pulses are transcutaneously delivered to the heart; and
a skin patch integrated with the transcutaneous cardiac stimulation device and the plurality of body-surface electrodes, the skin patch configured to be attached onto the skin such that electrical contacts between the pacing electrode set and the body allow for effective transcutaneous delivery of the pacing pulses to the heart.

2. The system of claim 1, wherein the skin patch comprises an attachment surface and an adhesive layer on the attachment surface, the attachment surface configured to be in contact with the skin through the adhesive layer during the transcutaneous delivery of the pacing pulses.

3. The system of claim 1, wherein the skin patch comprises means for pressing the body-surface electrodes against the skin to ensure that the electrical contacts between the pacing electrode set and the body allow for the effective transcutaneous delivery of the pacing pulses to the heart.

4. The system of claim 1, wherein the pacing control circuit is configured to control the transcutaneous delivery of the pacing pulses by automatically executing a pacing protocol, the pacing control circuit including a pacing protocol module and the pacing protocol stored in the pacing protocol module, the pacing protocol including a cardioprotective pacing protocol adapted to augment mechanical stress on the myocardium to a level effecting cardioprotection against myocardial injury using the pacing pulses.

5. The system of claim 4, wherein the pacing protocol comprises a cardioprotective pacing protocol specifying a pacing sequence including a specified number of cycles of alternating pacing and non-pacing periods, the pacing periods each specified as a pacing duration during which pacing pulses are programmed to be delivered, the non-pacing periods each specified as a non-pacing duration during which none of the pacing pulses is programmed to be delivered.

6. The system of claim 4, wherein the transcutaneous cardiac stimulation device comprises a monitoring circuit including a hemodynamic sensing circuit configured to sense an impedance signal indicative of hemodynamic performance using electrodes selected from the plurality of body-surface electrodes, and the pacing control circuit is configured to adjust the transcutaneous delivery of the pacing pulses using the sensed impedance signal.

7. The system of claim 4, wherein the transcutaneous cardiac stimulation device comprises a monitoring circuit including an electrocardiogram (ECG) amplifier circuit configured to sense one or more ECG signals using electrodes selected from the plurality of body-surface electrodes.

8. The system of claim 7, wherein the monitoring circuit comprises a capture verification circuit configured to determine whether each of the pacing pulses results in a cardiac depolarization and produce a capture verification signal indicative of a percentage of the pacing pulses resulting in the cardiac depolarizations.

9. The system of claim 8, wherein the pacing control circuit comprises one or more of:
a pacing energy adjustment module configured to adjust a pacing energy associated with the pacing pulses using the capture verification signal; and
an electrode selection module configured to select the pacing electrode set from the plurality of body-surface electrodes and adjust the selection of the pacing electrode set using the capture verification signal.

10. The system of claim 8, comprising an accelerometer integrated with the skin patch and configured to sense an acceleration signal indicative of a level of skeletal muscle contractions resulting from the pacing pulses delivered through the pacing electrode set, and wherein the pacing control circuit is configured to adjust the transcutaneous delivery of the pacing pulses using the capture verification signal and the acceleration signal.

11. The system of claim 7, wherein the monitoring circuit comprises an ischemia detection circuit configured to detect an ischemia event using the one or more ECG signals and produce an ischemia signal indicative of a detection of the ischemia event, and the pacing control circuit is configured to control the transcutaneous delivery of the pacing pulses using the ischemia signal.

12. The system of claim 11, wherein the ischemia detection circuit is configured to locate an ischemic region in the heart using a plurality of ECG signals sensed through a plurality of electrode pairs selected from the plurality of body-surface electrodes and produce an ischemia signal indicative of an approximate location of the ischemic region, and the pacing control circuit is configured to select the pacing electrode set from the plurality of body-surface electrodes based on the approximate location of the ischemic region.

13. The system of claim 4, wherein the pacing control circuit is configured to start executing the cardioprotective pacing protocol in response to a cardioprotective pacing command, and the transcutaneous cardiac stimulation device comprises a user interface configured to allow for starting, stopping, and adjusting the transcutaneous delivery of the pacing pulses, the user interface including a cardioprotective pacing button configured to receive the cardioprotective pacing command.

14. The system of claim 13, wherein the transcutaneous cardiac stimulation device comprises:
a defibrillation output circuit configured to deliver defibrillation pulses suitable for defibrillating the heart by transcutaneous delivery through a defibrillation electrode set including at least two electrodes selected from the plurality of body-surface electrodes; and
a defibrillation control circuit configured to control the delivery of the defibrillation pulses in response to a defibrillation command,
and wherein the user interface comprises a defibrillation button configured to receive the defibrillation command.

15. The system of claim 14, wherein the transcutaneous cardiac stimulation device comprises a tachyarrhythmia detection circuit configured to detect a specified type tachyarrhythmia and generate a tachyarrhythmia detection signal indicative of a detection of the specified type tachyarrhythmia, and wherein the defibrillation control circuit is configured to control the delivery of the defibrillation pulses in response to one of the defibrillation command and the tachyarrhythmia detection signal, and the pacing control circuit is configured to stop executing the pacing protocol in response to the one of the defibrillation command and the tachyarrhythmia detection signal.

16. A method for pacing a heart having a myocardium in a living body having a skin, the method comprising:
delivering pacing pulses transcutaneously to the heart from a transcutaneous cardiac stimulation device integrated with a skin patch through a pacing electrode set including two or more electrodes selected from a plurality of body-surface electrodes integrated with the skin patch, wherein the skin patch is attached onto the skin such that electrical contacts between the pacing electrode set and the body allow for effective transcutaneous delivery of the pacing pulses to the heart.

17. The method of claim 16, comprising attaching the skin patch onto the skin using an adhesive.

18. The method of claim 16, comprising attaching the skin patch onto the skin using one or more belts or straps.

19. The method of claim 16, comprising controlling the delivery of the pacing pulses by executing a cardioprotective pacing protocol adapted to augment mechanical stress on the myocardium to a level effecting cardioprotection against myocardial injury using the pacing pulses.

20. The method of claim 19, comprising:
sensing one or more electrocardiogram (ECG) signals using a monitoring electrode set including two or more electrodes selected from the plurality of body-surface electrodes;
determining whether each of the pacing pulses delivered through the pacing electrode set results in a cardiac depolarization using the one or more ECG signals;
producing a capture verification signal indicative of whether the each of the pacing pulses delivered through the pacing electrode set results in the cardiac depolarization; and
adjusting the delivery of the pacing pulses using the capture verification signal.

21. The method of claim 20, comprising receiving a muscular stimulation signal indicative of skeletal muscular contractions resulting from the pacing pulses, and adjusting the delivery of the pacing pulses using the capture verification signal and the muscular stimulation signal.

22. The method of claim 20, comprising:
detecting a specified type tachyarrhythmia using the one or more ECG signals;
stopping the execution of the cardioprotective pacing protocol in response to a detection of the specified type tachyarrhythmia; and
delivering a defibrillation pulse to the heart using a defibrillation electrode set including at least two defibrillation electrodes selected from the plurality of body-surface electrodes in response to the detection of the specified type tachyarrhythmia.

23. The method of claim 19, comprising:
locating an ischemic region in the heart;
producing an ischemia signal indicative of an approximate location of the ischemic region; and
controlling the delivery of the pacing pulses using the ischemia signal.

24. The method of claim 23, comprising selecting the pacing electrode set from the plurality of body-surface electrodes using the ischemic signal.

25. The method of claim 19, wherein executing the cardioprotective pacing protocol comprises delivering the pacing pulses according to a pacing sequence including a specified number of cycles of alternating pacing and non-pacing periods, the pacing periods each specified as a pacing duration during which pacing pulses are programmed to be delivered, the non-pacing periods each specified as a non-pacing duration during which none of the pacing pulses is programmed to be delivered.
